(19) United States
(12) Patent Application Publication
(10) Pub. No.: US 2007/0281071 A1
(43) Pub. Date: Dec. 6, 2007

(54) Acoustically coating workpieces

(75) Inventors: Thomas C. Eidenschink, Rogers, MN (US); James Q. Feng, Maple Grove, MN (US); James L. Shippy, Maple Grove, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC.

(21) Appl. No.: 11/447,172

(22) Filed: Jun. 6, 2006

(51) Int. Cl. A61L 33/00; B06B 1/00

(52) U.S. Cl. 427/2.1; 427/2.24; 427/600; 427/2.25

(57) ABSTRACT

The present invention is directed to methods, processes, and systems for acoustically coating portions of a workpiece as well as to workpieces that have themselves been acoustically coated in accord with the invention. In accord with the invention, for example, some or all outer surfaces of a workpiece, such as a medical implant, may be coated with a therapeutic while inner surfaces of the implant, which are not targeted for coating, may not be coated. Under methods and processes of the invention, a target surface of the workpiece may be identified. A droplet of therapeutic may then be generated with acoustic energy and deposited onto the target surface of the workpiece.

### TECHNICAL FIELD

The present invention generally relates to a workpiece that has been coated or otherwise treated in selected target areas by an acoustic coating system as well as to methods and systems for coating the workpiece in this fashion. More specifically, the present invention relates to acoustic coating systems, such as drop-on-demand systems, and methods and workpieces that have themselves been coated with these systems and methods. The workpieces of the present invention may be medical implants and the coating may comprise therapeutic.

### BACKGROUND

Coating workpieces is an often repeated procedure in contemporary manufacturing. Workpieces may be coated by methods that include tumble coating, spray coating, dip coating, and electrostatic spraying. During each of these procedures coating is applied to the workpiece prior to the workpiece being used for an intended purpose.

When the workpiece is formed partially or completely with a lattice framework or some other open structure, each of the faces of the struts that comprise the framework is exposed to coating during the coating process. By exposing each face of the workpiece to the coating being applied, each exposed face will be covered during the coating process.

When the workpiece being coated is an implantable medical device, such as a stent, all faces of the struts that comprise the stent are coated when using the coating systems identified above. For example, when dip coating is used, each face of the stent struts may be exposed to the coating. This coating will remain when the stent is removed from the dip and will dry on each face of the struts. Coating may also remain in the spaces between the struts. This phenomenon is sometimes called “webbing.” Here, not only are the individual struts covered, but some or all of the spaces between the struts are unnecessarily coated as well.

Further, in some applications, coating may also result in non-uniform application of the coating to the workpiece that can result in delamination and undercutting. Consequently, it may be more difficult to predict the dosage of therapeutic that will be delivered when the medical device is implanted.

### BRIEF DESCRIPTION

The present invention is directed to methods, processes, and systems for acoustically dispensing droplets, such as relatively fine droplets, for coating portions of a workpiece as well as to workpieces that have themselves been acoustically coated in accord with the invention. In the invention, for example, some or all outer surfaces of a workpiece, such as a medical implant, may be selectively coated with therapeutic while inner surfaces of the implant, which are not targeted for coating, may not be coated.

Under methods and processes of the invention, a dispensing device may be provided to apply therapeutic and/or another coating to targeted surfaces of the workpiece. These coating methods may include identifying a target area of a workpiece to coat, locating the target area to be coated in space, and using acoustic energy to generate and send droplets of coating towards the target areas. These drops of coating may be generated in a dispensing device, for example, an acoustic drop-on-demand dispensing device. A single layer of coating may be applied according to the present invention. Multiple layers of coating may also be applied with some or all of the layers being applied with the dispensing technology of the present invention. These layers may have uniform or varying thickness or even both. The workpiece may be an implantable medical device such as a stent or a vena-cava filter and the coating may comprise therapeutic.

The invention may be embodied in numerous devices and through numerous methods and systems. The following detailed description, which, when taken in conjunction with the annexed drawings, discloses examples of the invention. Other embodiments, which incorporate some or all of the features as taught herein, are also possible.

### BRIEF DESCRIPTION OF THE DRAWINGS

Referring to the drawings, which form a part of this disclosure:

FIG. 1a shows a dispensing device for applying a coating to a workpiece that may be employed in accord with the present invention;

FIG. 1b shows the formation of a droplet that has been formed in accord with the present invention;

FIG. 1c is a side-view in partial cross-section of the dispensing device of FIG. 1a that may be employed in accord with the present invention;

FIG. 1d shows a partial cross-section of another dispensing device that may be employed in accord with the present invention;

FIG. 2a is a cross-sectional view of a portion of a coated strut from a medical device that has been coated in accord with the present invention;

FIG. 2b is a cross-sectional view showing the coated strut of FIG. 2a after a second coating has been applied as may be employed in accord with the present invention;

FIG. 2c shows an arterial stent, which is a medical device that may be coated in accord with the present invention;

FIG. 3 shows a system for applying a coating to a medical implant using a dispensing device in accord with the present invention;

FIG. 4 shows a system for applying a coating to a medical implant using a dispensing device in accord with the present invention; and

FIG. 5 shows a system for applying a coating to an arterial stent positioned on a dilation member using a dispensing device in accord with the present invention.

### DETAILED DESCRIPTION

The present invention regards the selective coating of one or more surfaces of a workpiece with acoustic energy. In some embodiments this may include coating the outside or side surfaces of the workpiece while not coating the inside surfaces of the workpiece. By coating in this fashion the amount of coating resident on the workpiece may be selectively applied, such as in the drop-on-demand fashion. This can be useful when the amount of coating resident on the workpiece is metered or otherwise of interest. For example, if the workpiece is a stent and the coating contains therapeutic, a reduction in coating may allow the therapeutic to be delivered in a more targeted fashion after the stent is implanted at a target site. The limited use of coating can also conserve coating materials, which themselves may be valuable.

The selective coating of a workpiece may be accomplished in various ways in accord with the present invention. A dispensing device, which can generate droplets of therapeutic or other coating by using a focused acoustic wave or acoustic energy, may be used to direct coating at a surface of a workpiece to be treated. The acoustic wave is typically generated by a piezoelectric transducer and may be focused by an acoustic lens. A dispensing device, such as a drop-on-demand dispensing device, may include one or more piezoelectric transducers for generating multiple acoustic waves. This dispensing device may contain a pool of coating, such as a pool of liquid, that is impacted by the focused acoustic waves or acoustic energy generated by the device at a free surface of the liquid. The walls of the pool may be sized and shaped in certain ways such that these droplets may be generated with a desired firing rate, while the pool of coating may be unaffected by the orientation of the dispensing device. The pool of coating may also be sized to retard clogging or build up of the coating. For example, the opening 108 shown below may be greater than 100 microns, and may be preferably about 300 microns. This may be far greater than the 30-50 micron openings in non-acoustic systems, which may be prone to clogging as they are used. A lid with an opening sized to allow droplets of coating created by the acoustic wave or acoustic energy to move from the device toward the target can be added for coating evaporation control. The lid opening may be much larger than the droplets being created such that the droplet may pass through it without contacting the opening.

The dispensing device may be fluidly connected to a fluid reservoir storing or otherwise including coating that can be applied by the device. The device may also be in communication with a controller that also controls or at least receives signals from a calibration sensor that senses the presence and orientation of a workpiece to be coated. The piezoelectric transducers of the dispensing device, which are sources of acoustic waves, may be isolated from one another and may be individually addressable such that only one droplet may be created by the device at a time. Likewise, multiple droplets may also be created and fired at the workpiece depending upon the coating requirements.

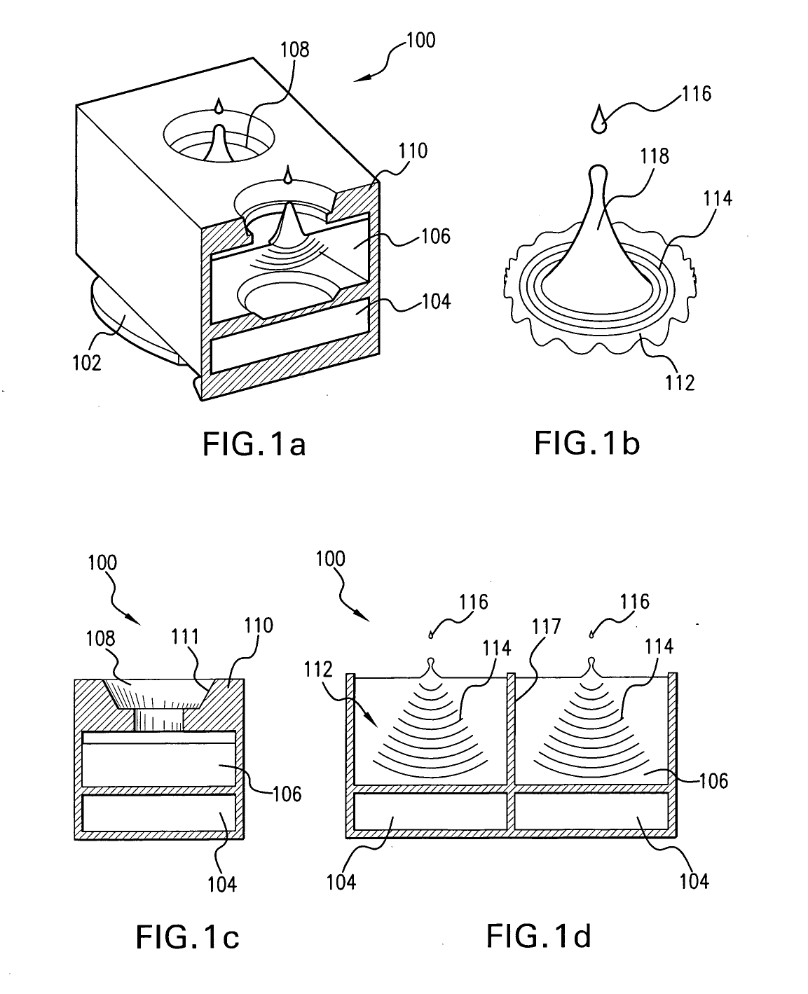

FIGS. 1a, 1c, and 1d show a dispensing device 100 in accord with the present invention. Evident in FIGS. 1a and 1c are a substrate 102, a piezoelectric transducer 104, a pool 106, and a cover 110 having pool walls 108. FIGS. 1a, 1c and 1d also show that the substrate 102, the transducer 104, the pool 106, and the cover 110 may be integrally connected and may be arranged in layers. For example, the transducer 104 may be positioned on top of the substrate 102, the pool 106 on top of the transducer 104, and the cover 110 on top of the pool 106. While the dispensing device 100 is shown as being rectangular, other shapes, sizes, and configurations may be used. The adjacent layers of the dispensing device can be secured to one another by various means and methods. For instance, they may be secured to one another with adhesives and may also be formed together during an injection molding process. The substrate 102 may be any suitable material including plastics, metals, or any combination thereof.

The transducer, which is used and controlled to generate acoustic waves, may be arranged on and in physical communication with the substrate 102. The transducer 104 in this example includes piezoelectric material which is used to generate an acoustic wave 114. The piezoelectric material of the dispensing device may contain many individual addressable regions that may be isolated from one another and may be controlled to allow placement or direction of coating by the dispensing device. A buffer material, which may be made of a suitable material such as metal, may be used between the transducer 104 and the pool 106 to purify the acoustic waves generated from the transducer prior to the waves reaching the coating. Thus, the buffer material can filter noise from the acoustic wave generated by the piezoelectric material.

An external voltage may be applied to the piezoelectric material of the transducer to activate it. When the voltage is applied at a certain frequency, the piezoelectric material may deform such that it produces mechanical vibrations that can emit acoustic waves through the buffer material into the coating. This wave of high and low pressure may carry energy that acts on the coating. Thus, when the wave is focused at a high enough intensity towards the coating, droplets may be created that pass from the dispensing device to the target area of the workpiece. Piezoelectric materials that may be used include quartz, rochelle salt, lead zirconate titanate, ceramics, barium titanate, tungsten-bronze, gallium orthophosphate, berlinite, polymer materials, and polymer films.

As noted, the transducer 104 may generate an acoustic wave that acts on the coating. The reaction of the coating upon encountering the acoustic wave is shown in FIG. 1b. As can be seen in FIG. 1b, the acoustic wave 112 may create a ripple 114, a cone 118, and a droplet 116 in the coating. The droplet 116 may be directed at the workpiece for coating the workpiece.

A coating 112 may be stored or otherwise included in the pool 106 of the dispensing device shown in FIGS. 1a, 1c, and 1d. The pool 106 may be self-contained or may also fluidly communicate with an external fluid source and/or passage. The pool 106 may be partially enclosed by the cover 110. A surface contour 111 may extend through the thickness of the cover 110 to form the opening 108. Alternatively, as seen in FIG. 1d, the opening 108 may be defined by the boundaries of the pool 106. In FIG. 1d, two pools 106 are provided which are filled with fluid 112. The pools 106 may be divided by a wall 117. The wall 117 may eliminate or reduce crosstalk between adjacent pools 106. Likewise, the cover 110 and/or side walls of the pool 106 may also eliminate or reduce crosstalk. As the acoustic wave reaches the fluid 112 surface, local pressure from the focused beam pushes on a portion of the fluid 112. Thus, as seen in FIG. 1b, the pressure causes a droplet 116 to break away from the surface of the fluid.

An aerosol mist, or a combination of mist and a droplet 116, may also be generated by the dispensing device. For instance, the dispensing device 100 can generate droplets of volumes from about 1 femtoliter (fL) or 10⁻¹⁵ to 10 nanoliter (nL) or 10⁻⁹. At around 1 fl, an aerosol mist may be formed. At around 2 pL, a droplet may form. The characteristics of the droplet 116 may depend, in part, on the firing pulse and firing rate of the dispensing device 100. If operated at a 25 kHz firing rate a 16 pL droplet 116 may form. If operated at a 50 kHz firing rate a 2 pL droplet 116 may form. Further, if operated at 2 kHz firing rate a mist of 1 fL may form. The acoustic wave may range from about 50-300 MHz. In the example, both aqueous and non-aqueous solutions 112 may be used.

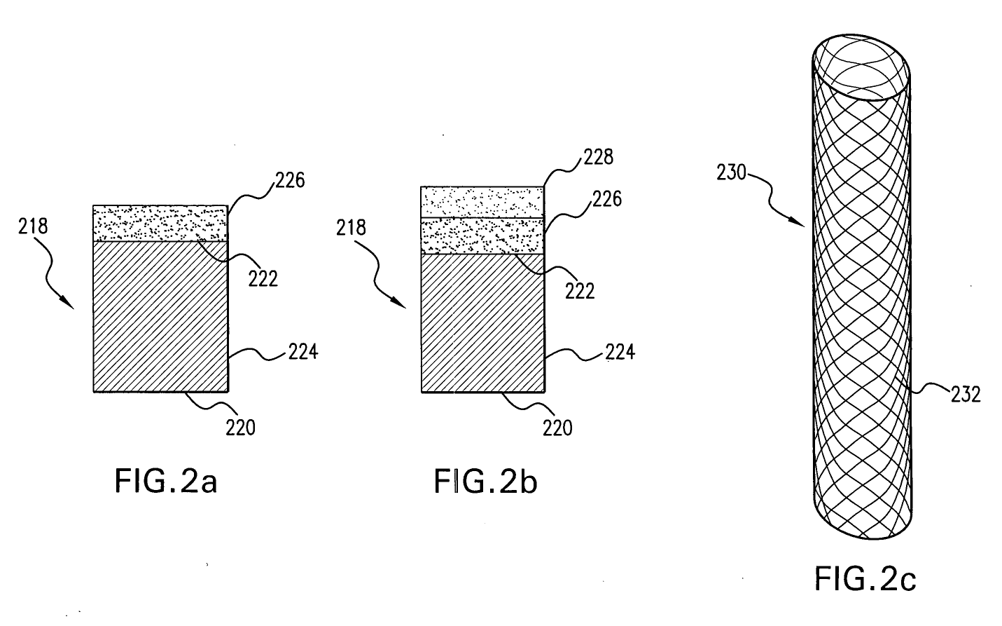

FIG. 2a is a side sectional view of a strut 218 of a stent 230 that may be coated in accord with the present invention. The strut 218 in FIG. 2a has an inner surface 220, an outer surface 222, and two cut faces 224. Also shown on the strut 218 is a coating 226. As can be seen, the coating 226, covers only one face of the strut 218. As shown in subsequent illustrations, this stent strut may be coated by the dispensing device by placing the stent in a coating chamber, locating the stent in the chamber, and sending coating at the stent while the coating device is moved about the stent. In this fashion, the location of the strut to be coated would be sensed or otherwise previously located and then coated with individual droplets of coating acoustically generated by the dispensing device moving about the stent.

FIG. 2b shows another example of how a coating may be applied in accord with the invention. In FIG. 2b, a first coating 226 and a second coating 228 have been applied to the strut 218. As can be seen, the first coating 226 is in contact with the strut 218 while the second coating 228 is in contact with the first coating 226 and further covers the outer surface 222 of the strut 218. This second coating 228 may be applied in accord with the processes and methods of the present invention. It may also be applied with different methods and processes. In this example, as well as with the others described herein, if a second coating 228 is employed this coating may comprise the same materials as the first coating 228 and it may differ from the materials used for the first coating 226. In still other examples the coating may be applied in other patterns as well. For example, it may be applied to opposing cut faces 224 and not the outer surface 222, likewise it may be applied to both cut faces 224 and the outer surface 222. In a exemplary embodiment, the outer surface 222 is coated and the two cut faces 224 as well as the inner surface 220 are not.

FIG. 2c is a side view of an implantable aortic stent 230 including a lattice portion 232 that may be coated in accord with the invention. The stent 230 may be porous or have portions thereof that are porous. The struts 218 shown in FIGS. 2a and 2b are struts that may comprise and make up this stent 230. While the workpiece 230 shown in these initial figures is a stent, many other workpieces 230 may be coated in accord with the invention. For example, and as mentioned above, other medical devices that may be coated include filters (e.g., vena cava filters, stent grafts, vascular grafts, intraluminal paving systems, implants and other devices used in connection with drug-loaded polymer coatings). Likewise, the workpeice 230 may not be an implantable medical device but may, instead, be another piece that needs to be coated only on certain pre-selected surfaces. In some instances these medical devices or other workpieces 230 may be made from conductive materials and in other instances they may not be. Nonconductive materials may include polymers and ceramics.

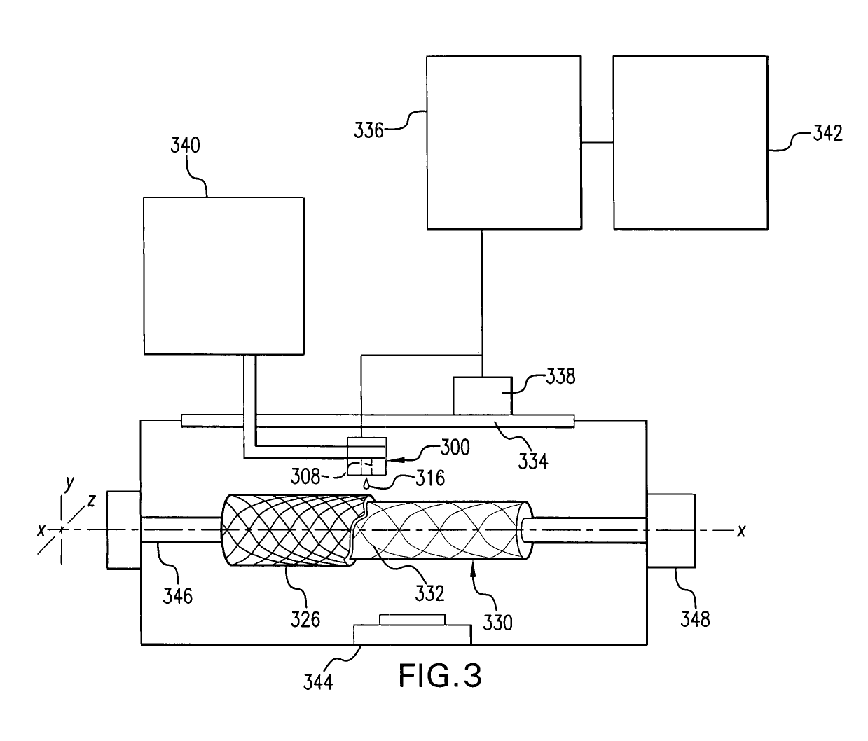

FIG. 3 illustrates a system for coating a medical implant 330 using a dispensing device 300 in accord with the present invention. In this system, the dispensing device 300 may be used to force coating 326 onto the outer surface 332 of medical implant 330. As shown, the dispensing device 300 may be placed in close proximity to the medical implant 330 and may be moved longitudinally (with respect to the x-axis of the medical implant 330) along track 334 so that it may apply coating 326 to separate discrete spots or portions of the outside surface 332 of the medical implant 330. In other words, as the dispensing device 300 moves back and forth along track 334, the dispensing device 300 may force coating onto the medical implant 330. It may do this while concurrently refraining from forcing coating 326 into spaces between the struts forming the outer surface 332, because coating 326 forced into these spaces would simply be wasted or result in errant deposits of coating 326 elsewhere on the medical implant 330. As can be seen in FIG. 3, a portion of the medical implant 330 has already been coated 326, while another portion of the medical implant 330 has not been coated. FIG. 3 also illustrates droplets of fluid 316 being ejected from the dispensing device 300.

The dispensing device 300 may be controlled by, or at least receive signals from, an electronic signal processor 336, which may instruct the dispensing device 300 to selectively coat portions of the medical implant 330. For example, in FIG. 3, only the outer surface 332 of the medical implant 330 may be coated. When coating or at some other time, the processor 336 may receive signals from a sensor that reads the presence and location of the workpiece to be coated. It may also read the surface configuration of the workpiece. By knowing this information, the controller may control where the coating may be applied using the physical location of the workpiece and the pattern chosen for coating. In other words, the processor may align the coating schedule with the location of the workpiece in order to place the coating on the workpiece as intended rather than to simply miss the workpiece or apply coating in the wrong place because the exact location of the workpiece is not accurately known.

The dispensing device 300 may be adjustable to increase or decrease the distance between the opening 308 and the medical implant 330. The distance between the opening 308 and the medical implant 330 may depend on factors such as the diameter of the opening 308 and the viscosity of the fluid 316 being applied to the medical implant 330. As stated, an exemplary range of values for the diameter of the opening 308 may be from about 100 microns to at least 300 microns.

The track may be controlled by a drive mechanism 338. The drive mechanism 338 can be use an electrical, mechanical, or hydraulic drive source, and may use combinations thereof. For example, motors, endless belts, gearing, or hand cranks may be used.

In FIG. 3, an external fluid source 340 is shown in fluid communication with the dispensing device 300 and may be used to supply fluid 316 to the dispensing device 300. Also, a storage media 342 may be in communication with processor 336 and may be used to store and provide instructions for processor 336 and coating source 340 for coating 326 the medical implant 330. Storage media 342 may be one of numerous types of available storage media including both volatile (i.e. RAM) and non-volatile storage devices (i.e. ROM, CD ROM, EEPROM, Magnetic Media, etc.). The pre-programmed instructions or other retained data may be unique to each medical implant 330. The processor 336 and storage media 342 may communicate with a sensor 344 to account for each unique external pattern and precise dimensions of each medical implant 330 that may be coated by the dispensing device 300. Storage media 342 may also hold unique instruction sets for many different medical implants 330, or may be provided with a media receptacle such as a disk drive that accommodates different recordable media, each recordable media holding a unique instruction set for a single medical implant or a set of instructions for multiple medical implants.

Also as seen in FIG. 3, the medical implant 330 may be rotated by a holder 346 in order to expose different sides of the medical implant 330 to the acoustic type dispensing device 300. The holder 346 may include a mandrel. The mandrel can be made of any material that may reduce movement of the medical implant 330 and a material that may be visually different than that of the medical implant 330. The holder 346 may be rotatably connected to an electric, mechanical, or hydraulic drive source 348 or may be combinations thereof. Thus, through the coordinated movement of the acoustic type dispensing device 300 on track 334 and the medical implant 330 on holder 346, all of the outside surface 332 of medical implant 330 may be exposed to and coated by the droplets of fluid 316 ejected from the acoustic type dispensing device 300.

In other arrangements, wherein the medical implant 330 may be orientated differently, such as vertically, the configurations may be different than that described above. As stated herein, in these configurations, the support structure may provide for movement of the medical implant 330 in both the x, y, and z planes or around the diameter of the medical implant 330 such that the dispensing device 300 may move around the entire outside surface of the medical implant 330.

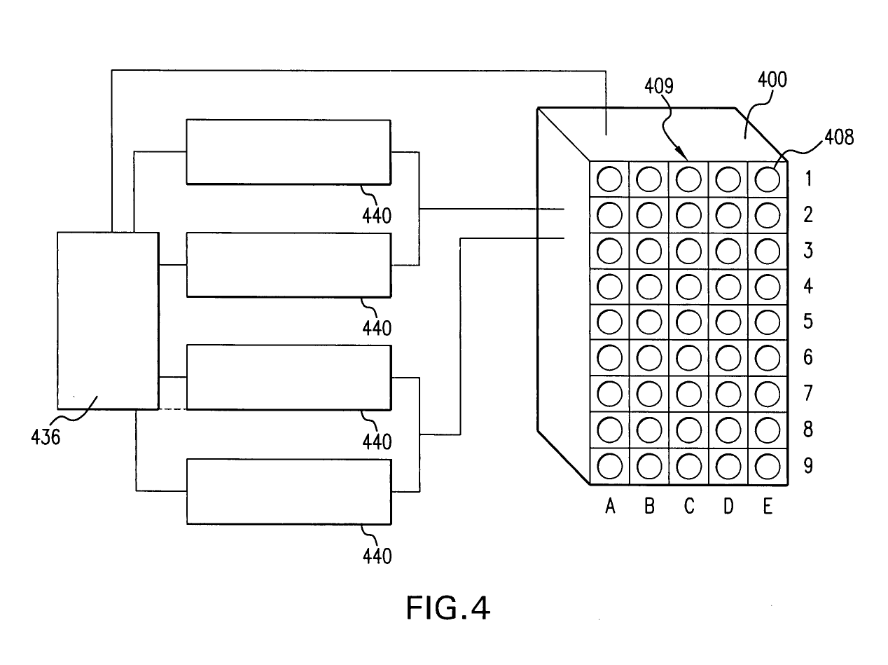

FIG. 4 shows that the dispensing device 400 may be in fluid communication with a plurality of fluid sources 440. This figure also shows that the dispensing device 400 can include a plurality of openings 408 forming a matrix 409. The matrix 409 may be used to coat multiple workpieces or a single workpiece. The dispensing device 400 may also be connected to a processor 436 as described elsewhere.

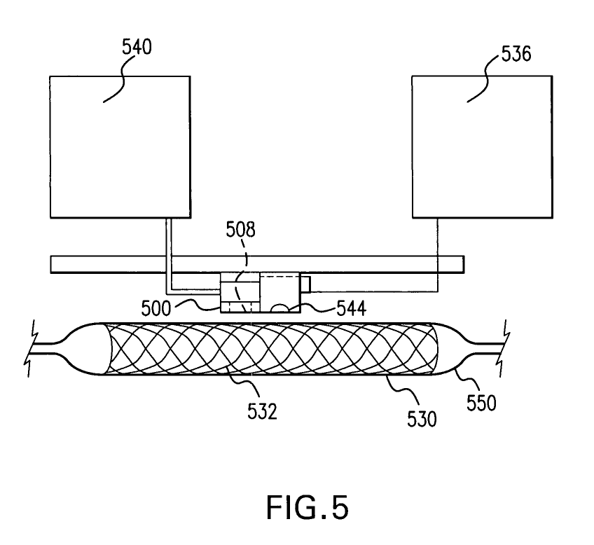

FIG. 5 shows a system for coating a stent 530 using a dispensing device 500 communicating with a fluid source 540 in accord with the present invention. In this embodiment, the stent 530 is shown positioned on a dilation member 550. In the example, the stent 530 may be crimped onto the dilation member 550. Due to the relative precision of the coating process utilizing a dispensing device 500, such preassembly of components may be plausible within the embodiments of the invention.

Also shown in FIG. 5, a visual detection member 544 may be used for calibrating the dispensing device 500. The visual detection member 544 may communicate with the processor 536 to control the movements of dispensing device 500, the rotation of the stent 530 and dilation member 550, and the droplet ejection from the dispensing device 500.

The visual detection member 544 may be used in any one of, or any combination of, the following ways. First, the visual detection member 544 may be used to determine the position and orientation of the stent 530 by scanning an identifiable feature on the stent 530 such as a strut 532 before or during coating. Second, the visual detection member 544 may be used to determine the position and orientation of the opening 508 of the dispensing device 500 by observing the dispensing device 500 directly before or during coating. Third, the visual detection member 500 may be used to monitor the amount coating on the stent 530 as the coating process proceeds. The visual detection member 544 may be any number of systems including optics, laser scanners, video cameras, visible light scanners, and invisible light scanners.

It should be understood that the foregoing descriptions of various examples of the dispensing device, support structure, and workpiece are not intended to be limiting, and any number of modifications, combinations, and alternatives of the examples may be employed to facilitate the effectiveness of the coating of target surfaces of the workpiece.

As described above, the dispensing device may be in fluid communication with a fluid source. The fluid source may contain any one of several possible coatings to be placed on the workpiece.

The coating, in accord with the embodiments of the present invention, may comprise a polymeric and or therapeutic agent formed, for example, by admixing a drug agent with a liquid polymer, in the absence of a solvent, to form a liquid polymer/drug agent mixture. A suitable list of drugs and/or polymer combinations is listed below. The term “therapeutic agent” as used herein includes one or more “therapeutic agents” or “drugs”. The terms “therapeutic agents” or “drugs” can be used interchangeably herein and include pharmaceutically active compounds, nucleic acids with and without carrier vectors such as lipids, compacting agents (such as histones), viruses (such as adenovirus, andenoassociated virus, retrovirus, lentivirus and α-virus), polymers, hyaluronic acid, proteins, cells and the like, with or without targeting sequences.

Specific examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor ∀ and ∃, platelet-derived endothelial growth factor, platelet-derived growth factor, tumor necrosis factor ∀, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

As stated above, coatings used with the exemplary embodiments of the present invention may comprise a polymeric material/drug agent matrix formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

In accord with the embodiments, the polymer used to coat the medical device is provided in the form of a coating on an expandable portion of a medical device. After applying the drug solution to the polymer and evaporating the volatile solvent from the polymer, the medical device is inserted into a body lumen where it is positioned to a target location. In the case of a balloon catheter, the expandable portion of the catheter is subsequently expanded to bring the drug-impregnated polymer coating into contact with the lumen wall. The drug is released from the polymer as it slowly dissolves into the aqueous bodily fluids and diffuses out of the polymer. This enables administration of the drug to be site-specific, limiting the exposure of the rest of the body to the drug.

The polymer used in the exemplary embodiments of the present invention is preferably capable of absorbing a substantial amount of drug solution. When applied as a coating on a medical device in accordance with the present invention, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present invention to apply multiple layers of polymer coating onto a medical device. Such multiple layers are of the same or different polymer materials.

The polymer of the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

What is claimed is:

1. A method of coating a surface of a workpiece with therapeutic, the method comprising:
identifying a target surface of the workpiece;
generating a droplet of therapeutic coating with acoustic energy; and
depositing the droplet of coating onto the target surface of the workpiece.

2. The method of claim 1, wherein the dispensing device includes a transducer including piezoelectric material to generate at least one acoustic wave.

3. The method of claim 1, wherein the target surface is an outer surface of a medical implant.

4. The method of claim 1, further comprising positioning the workpiece on a holder.

5. The method of claim 1, further comprising using a sensor to locate the position of the workpiece prior to ejecting therapeutic coating from the dispensing device.

6. The method of claim 1, wherein the dispensing device comprises a plurality of orifices having an opening of at least 100 microns.

7. The method of claim 1, wherein the dispensing device contains a piezoelectric layer, a pool, and a top layer.

8. The method of claim 1, wherein the acoustic energy is in the form of an acoustic wave.

9. The method of claim 1, wherein the acoustic energy is filtered before ejecting the therapeutic coating from the dispensing device.

10. The method of claim 9, wherein the dispensing device is in the form of a rectangle with an array of apertures on one of its faces.

11. A method of coating a surface of a medical implant, the method comprising:
providing a dispensing device;
identifying a target surface of the medical implant; and
sending coating to the target surface after generating an acoustic wave.

12. The method of claim 11, wherein the dispensing device includes a transducer including piezoelectric material to generate at least one acoustic wave.

13. The method of claim 11, wherein the target surface is an outer surface of the medical implant.

14. The method of claim 11, wherein the dispensing device is moveable about the medical implant.

15. The method of claim 11, wherein the dispensing device includes a plurality of dispensing devices.

16. The method of claim 11, wherein the dispensing device has a plurality of openings.

17. The method of claim 11, wherein the dispensing device includes an opening.

18. The method of claim 17, wherein the opening is about 300 microns.

19. The method of claim 11, further comprising providing a visual detection member to calibrate a position of the dispensing device or the medical implant.

20. A method of coating a stent, the method comprising:
providing a stent having a lattice portion;
providing a dispensing device including a transducer to generate at least one acoustic wave; and
positioning the dispensing device such that coating is transferred from the dispensing device to an outside surface of the lattice portion after an acoustic wave is generated by the transducer.

21. The method of claim 20, further comprising providing a visual detection member to calibrate a position of the dispensing device or the stent.

22. The method of claim 20, further comprising positioning the stent over a dilation member.

23. The method of claim 20, wherein the dispensing device is moveable about the stent.

24. The method of claim 20, wherein the dispensing device includes a plurality of dispensing devices.

25. The method of claim 20, wherein the dispensing device has a plurality of openings forming a matrix.

26. The method of claim 20, wherein the dispensing device is in fluid communication with a therapeutic coating source.
